(19) United States
(12) Patent Application Publication
(10) Pub. No.: US 2023/0149660 A1
(43) Pub. Date: May 18, 2023

(54) DEVICE FOR GENERATING SENSORY STIMULATION FOR A CHILD

(71) Applicant: Natus Medical Incorporated, Orlando, FL (US)

(72) Inventors: Raphael Patrick Manfred LANG, Aictal (DE); Camilo Andres ANABALON ALAMOS, Aictal (DE); Maximilian BLENDINGER, Aichtal (DE)

(73) Assignee: Natus Medical Incorporated, Orlando, FL (US)

(21) Appl. No.: 17/754,708

(22) PCT Filed: Oct. 12, 2020
(86) PCT No.: PCT/DE2020/100883

§ 371 Date: Apr. 8, 2022

(30) Foreign Application Priority Data
Oct. 10, 2019 (DE) 102019127394.3

(51) Int. Cl. A61M 21/02

(52) U.S. Cl. CPC A61M 21/02; A61M 2021/0022

(57) ABSTRACT

A device for producing sensory stimuli for a child by means of a body made of an elastic material and a bellows which is embedded in the elastic material of the body, including a first converter device for generating a fluid flow for moving a lying surface for a child, a second converter device for generating sound in a fluid, which corresponds at least to either heart sounds or human speech or music, and a fluid line for simultaneously conducting the fluid flow generated by the first converter device and transmitting the sound generated by the second converter device to a bellows in a body made of an elastic material.

### RELATED APPLICATIONS

This application claims priority under 35 U.S.C. § 371 of PCT Application No. PCT/DE2020/100883, entitled Device for Generating Sensory Stimulation for a Child filed Oct. 12, 2020, the content of which is incorporated herein in its entirety.

### FIELD OF THE INVENTION

The present invention relates to a device for generating sensory stimulation for a child in an incubator.

### BACKGROUND OF THE INVENTION

The present invention is directed to an emulation or simulation of sensory stimuli, which originate from a reference person, for a child which is spatially spaced apart from the reference person. In particular, the present invention is directed to forward directions, a system and a method for generating sink stimuli corresponding to the sensory stimuli emanating from a reference person for a child.

The birth represents a drastic change in all of its operating conditions for a person. The direct physical contact of the newborn with its nut, at whose heat, odor, voice, movements and heartbeat it is already accustomed, is therefore particularly important and valuable for it. For medical and other reasons, however, this direct physical contact is not always possible. In particular after an early birth, the newborn must often spend many weeks in an incubator.

An incubator or incubator represents a hygienic room with an advantageous microclimate for a newborn, in particular with optimum temperature and humidity. However, the sensory stimuli which are familiar to the newborn from the time prior to its birth are missing, in particular the movements, the voice, the heart sounds and the odor of the nut. There are numerous approaches for subjecting an infant or other human to corresponding sensory stimuli.

U.S. Pat. No. 3,672,354 describes a device having an inflatable cushion. An air compressor generates pressure pulses at the frequency of the human heart.

U.S. Pat. No. 3,809,065 describes a system for subjecting a newborn slowly changing environmental conditions. The temperature, tactile perception, noises, movement and light are slowly different from the conditions prevailing in the womb, up to the conditions prevailing outside the womb.

In US 2011/0144416 A1, a device is described for generating rhythmical movements similar to those experienced by a neonate.

WO 2011/063077 A1 describes a device 10B, 10C for simulating sound and movements in the uterus (paragraph [0056]; FIGS. 3A and 5). The front direction comprises a sealed outer casing 14 on a flat surface of a support element 60 (paragraph [0062], FIG. 3A). A movement activity unit 42 is arranged within the sealed outer casing 14 (implicit in paragraph [0058], FIG. 3A). The movement activation unit 42 comprises aisle or body movement bellows or cushions 44 and a breathing bladder or a breathing cushion 46 (paragraph [0058], FIG. 3A).

GB 2 359 994 A describes a device for simulating the heartbeat of a mut for a child lying in a bed (page 1, lines 12 and 13). The device comprises a hollow flexible bladder 14 which is arranged between a mattress 10 and a ceiling 12 (page 4, lines 9 to 11; FIGS. 1 and 2).

EP 1 473 013 A2 describes a vibrating patient support system having a bladder 14 and a vibration cushion 12 in a cushion device 10 (paragraphs [0007], [0017]). The vibration cushion 12 is arranged below the lower surface of the bladder 14 or within the bladder 14 and below the upper surface of the bladder 14 (paragraphs [0007], [0017]).

U.S. Pat. No. 7,475,441 B1 describes a baby carrier device 10 on which a baby can lie (column 3, lines 21 and 22). The device 10 comprises a platform 18 and an elastic padding 16 on the upper side of the platform 18 (column 3, lines 28 to 36). A hinge 22 connects a first end of the platform 18 to a base 20 (column 3, lines 47 and 48). A moving cylinder 30 moves the second end of the platform 18 relative to the base 20 (column 3, lines 48 to 51).

In DE 10 201 2 103 862 A1, EP 2 659 929 A2, US 2014/0330070 A1 and EP 3 202 450 A1 describes devices for generating sensory stimuli which correspond to the sensory stimuli emanating from a reference person for a child. A first device comprises a sensor for detecting heart sounds and/or chest movements of the reference person and a transmission device for transmitting a data stream which describes the heart sounds and/or the chest movements. A second device comprises an EMP device for receiving the data stream, a device for producing sound, which corresponds to heart sounds described by the data stream, and a device for generating movements which correspond to the movements described by the data stream. The device for generating sound is connected by a first fluid line to a bellows in a body made of an elastic material which has a lying surface for a child. The device for generating movements is connected to a further bellows in the body by means of a second fluid line.

An object of the present invention is to provide an improved device for producing sensory stimuli which correspond to the sensory stimuli originating from a reference person.

This problem is solved by the subject matter of the independent claims. Developments are specified in the dependent claims.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to a device for producing sensory stimuli for a child by means of a body made of an elastic material and a bellows which is embedded in the elastic material of the body. Said device comprises a first converter device for generating a fluid flow for moving a lying surface for a child of a second converter device for generating sound in a fluid which corresponds to at least either heart sounds or human speech or music and a fluid line for simultaneously conducting the fluid flow generated by the first converter device and transmitting the sound generated by the second converter device to a bellows in a body made of an elastic material.

The first converter device and the second converter device are provided and designed in particular for permanent arrangement outside an incubator or incubator for an early child. Only the fluid line alone connects the transducer devices outside the incubator to one or more bellows in a body made of an elastic material within the incubator. This body can also be referred to as a mattress. An upper surface region of the body forms a lying surface for a child in the incubator.

The one and only fluid line can be laid in a particularly simple manner and occupies only a relatively small installation space. In particular, it can be laid more easily and occupies a smaller installation space than two or more fluid lines, as described in DE 10 201 2 103 862 A1, EP 2 659 929 A2, US 2014/0330070 A1 and EP 3 202 450 A1. In this case, however, this one fluid line transmits not only a fluid which causes a vertical movement of the lying surface via the bellows or bellows in the elastic material of the body. At the same time, the one fluid line transmits fluid vibrations or sound and thus heart sounds and/or the voice of a parent part or another reference person of the child and/or music or other noises to the body and thus to the lying surface on which the child lies.

A device as described here further comprises, in particular, a fluidic connection device with three connections for simultaneously connecting the first converter device and the second converter device to the fluid line, wherein a first connection of the fluidic connection device to the first converter device, a second connection of the fluidic connection device to the second converter device, and a third connection of the fluidic connection device to the fluid line are fluidically connected.

In a device as described here, the fluidic connection device comprises in particular a pipe connection piece with three connections. The pipe connection piece is in particular a pipe-T-piece or a V- or star-shaped pipe piece with three connections.

A device as described here further comprises, in particular, a first line section, which connects the first converter device to a first connection of the pipe connection piece, and a second line section, which connects the second converter device to a second connection of the pipe connection piece arranged symmetrically to the first connection of the pipe connection piece, wherein a third connection of the pipe connection piece is connected to the fluid line.

In a device as described here, the first converter device comprises in particular an electromotively driven bellows, wherein the second converter device comprises a loudspeaker. The bellows is driven in particular by a stepping motor and via a spindle drive or by a linear motor.

In a device as described here, the loudspeaker comprises in particular an electromagnetically or electrostatically or electrostrictive membrane, the edge of which is connected in a fluid-tight manner to an edge of a sound funnel. The horn is a funnel-shaped or conical or cylindrical tubular or dome-shaped or dome-shaped component. The edge at one end of the sound funnel is connected to the edge of the membrane directly or indirectly in a fluid-tight manner. The other end of the horn has, in particular, a small diameter than the membrane and is connected to the pipe connection piece via the second line section.

A device as described here further comprises, in particular, a third converter device for generating sound in a fluid, which corresponds at least to either human speech or music, wherein the second transducer device is provided and designed to generate sound corresponding to heart sounds wherein the fluid line is provided and designed for simultaneously conducting the fluid flow generated by the first converter device and transmitting the sound generated by the two converter device and transmitting the sound generated by the third converter device. The second converter device and the third converter device can be identical or have different properties.

A device as described here further comprises, in particular, a fluidic connection device with four connections for simultaneously connecting the first converter device and the second converter device and the third converter device to the fluid line, wherein a first connection of the fluidic connection device to the first converter device, a second connection of the fluidic connection device to the second wall, a third connection of the fluidic connection device is fluidically connected to the third converter device and a fourth connection of the fluidic connection device is fluidically connected to the fluid line.

In a device as described here, the fluidic connection device comprises in particular a first pipe connection piece and a second pipe connection piece, wherein the first pipe connection piece connects the first converter device and the second pipe connection piece to the fluid line, and the second pipe connection piece connects the second converter device and the twisted converter device to the first pipe connection piece. The first pipe connection piece and the second pipe connection piece can each be designed as a pipe-T piece or as a V-shaped or star-shaped pipe piece.

A device as described here further comprises, in particular, a body made of an elastic material, a lying surface for a child on the body and a bellows which is embedded in the elastic material of the body, wherein the bellows is provided and designed to generate a vertical movement of the lying surface and to transmit sound from a fluid into the elastic material of the body, wherein the bellows is or can be connected to the converter devices through the fluid line.

A device for producing sensory stimuli for a child comprises a body made of an elastic material, a lying surface for a child on the body and a bellows which is embedded in the elastic material of the body, wherein the bellows is designed and designed to generate a vertical movement of the lying surface and to transfer sound from the fluid into the elastic material of the body. The body with the lying surface and the bellows forms, in particular, a mattress for arrangement in an incubator. The bellows or a plurality of bellows are provided and designed in particular for generating a vertical movement, which corresponds to a movement of the front side of the upper body of a nut or of another adult human due to respiration, and for generating structure-borne sound which corresponds to the heartbeat and/or the voice of a nut or another adult human. The structure-borne sound is expressed in particular in vertical movements of the lying surface, which can separate from movements corresponding to breathing movements in the frequency, the amplitude, the spatial distribution and the time dependence. The body has no further cavities, in particular, apart from the bellows or from a plurality of bellows. By virtue of the fact that the body does not have any further cavities, contamination of cavities and a colonization of cavities with germs are exhausted. The device can therefore be particularly easy to clean and to sterile.

A device having a body made of an elastic material, as described here, is produced in particular by a casting method in which the elastic material of the body is integrally bonded to the elastic film and to the bellows over the entire surface or substantially over the entire surface. In particular, two bellows are arranged symmetrically or substantially symmetrically in the body in order to simulate the lungs of the human body.

A device for generating sensory stimuli for a child comprises an EMP capturing device for receiving data describing sound, and a transducer device for generating sound, which corresponds at least to either heart sounds or human speech or music, in a body made of an elastic material which has a lying surface for a child. The EMP device is provided and designed to receive analog or digital data from a mobile telephone or other mobile communication device or from a tablet computer or another portable computer. The receiving device is designed in particular for receiving data on optical, optical or electromagnetic paths. The receiving direction comprises, for example, a plug-in connector for electrically receiving analog or digital data or for optically receiving analog or digital data. Alternatively or additionally, the receiving device can have an antenna for receiving electromagnetic waves.

The device for generating sensory stimuli may further comprise a surface area for receiving a mobile phone or other mobile communication device or a tablet computer or other portable device. The surface region is in particular a planar or continuous surface region on an upper side or near an upper side of the device for producing sensory stimuli. For example, the surface region is designed as a trough, the size and shape of which is adapted to the shape of a mobile telephone or another mobile communication device or of a tablet computer or of another portable computer.

A system comprises a device for detecting sensory stimuli with a sensor at least either for detecting heart sounds of a reference person or for detecting chest movements of the reference person and for detecting data which the heart sounds or chest movements may be simulated.

the invention relates to a device for generating sensory stimuli, which correspond to the sensory stimuli emanating from the reference person, for a child, and a controller, wherein the device for generating sensory stimuli comprises a receiving device for receiving data from the device for detecting sensory stimuli and a converter device which is coupled to the receiving device and can be controlled by the data wherein the transducer device is designed to generate at least either structure-borne sound in a body made of an elastic material with a lying surface for the child or vertical movements of the lying surface of the body, wherein the system further comprises a sensor for detecting at least one orientation of the device for detecting sensory stimuli in the space or a position of the device for detecting sensory stimuli relative to the device for producing sensory stimuli, wherein the controller is designed to control, depending on the detected orientation or position, a transmission of data from the device for detecting to the device for generating.

The device for producing sensory stimuli can be a device for producing sensory stimuli, as described above. In particular, the device for generating sensory stimuli comprises one or more bellows embedded in a physical material of the body and/or a converter device for generating a fluid flow for moving a lying surface for a child and/or for generating sound in a fluid which corresponds at least to either heart sounds or human speech or music, wherein the fluid flow and/or the sound in the fluid is transmitted through a fluid line to a body made of an elastic material and are converted by one or more bellows in the elastic material into a vertical movement of the bed surface of the body or into sound in the elastic body.

The controller can be arranged completely in the device for detecting sensory stimuli or completely in the device for producing sensory stimuli. Alternatively, the controller can be partially arranged in the device for detecting sensory stimuli and partially in the device for producing sensory stimuli. The controller is designed, in particular, to start the transmission of data from the device for detecting sensory stimuli to the device for generating sensory stimuli when the device for detecting sensory stimuli is arranged in the immediate vicinity of the device for generating sensory stimuli and in a predetermined orientation in the space. The predetermined orientation is in particular a vertical or substantially vertical orientation. The predetermined orientation and/or the predetermined positioning of the device for detecting sensory stimuli can be defined by a positioning device on the device for generating sensory stimuli. The positioning device comprises, for example, a trough or another concave surface region on the device for producing sin cavities and/or a fastening device for the releasable mechanical fastening of the device for detecting sensory stimuli on the device for producing sensory stimuli. The fluid is in particular air or nitrogen.

### BRIEF DESCRIPTION OF THE DRAWINGS

Some embodiments of the present invention are illustrated as an example and are not limited by the figures of the accompanying drawings, in which like references may indicate similar elements.

FIG. 1 shows a schematic axonometric representation of a system.

FIG. 2 is a schematic axonometric representation of the transducer device of the system.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

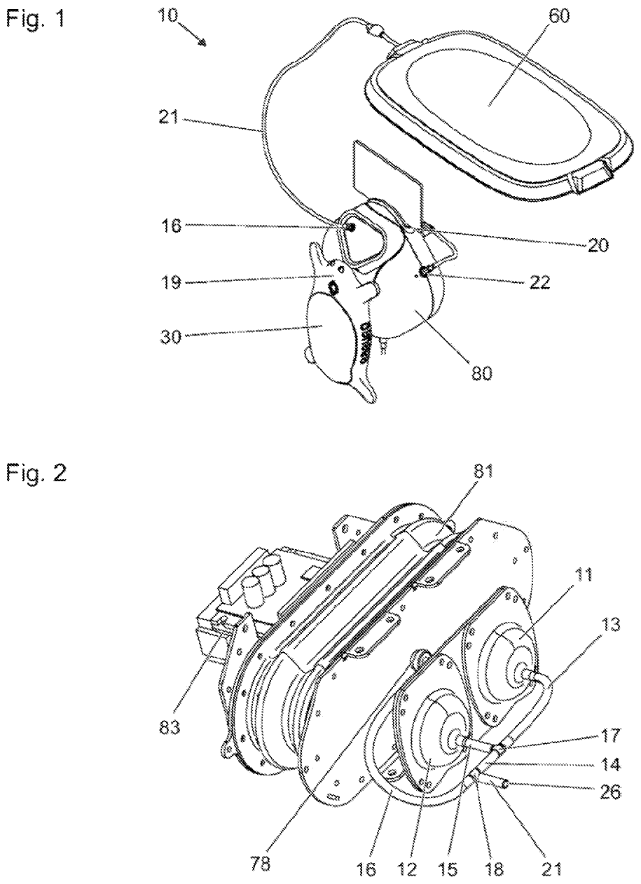

FIG. 1 shows a schematic axonometric representation of a system 10 with a device 30 for detecting sensory stimuli generated by a nut or another reference person of a child, and a forward direction 80, 60 for generating sensory stimuli for the child. The device 30 for detecting sensory stimuli is designed to be carried by the reference person on the body, in particular on the upper body in the region of the thorax. For this purpose, the device 30 for detecting can have one or more straps or fastening means (not shown in FIG. 1) for one or more belts, which enable a releasable and as comfortable as possible fastening of the device 30 to the upper body.

The device 30 for detecting sensory stimuli comprises one or more sensors (not shown in FIG. 1) for detecting movements of the upper body, in particular of the thorax of the reference person, as are caused in particular by respiration. Alternatively or additionally, the sensor (s) can generally be designed to detect all movements of the reference person or the upper body of the reference person.

Furthermore, the device 30 for detecting sensory stimuli comprises one or more sensors (not shown in FIG. 1) for detecting heart sounds of the reference person and noises which the reference person generates, in particular with their voice. These sensors comprise, in particular, microphones which are suitable for the described tasks due to the frequency dependence of their sensitivity and their arrangement. In particular, heart sounds are never more frequent and can be detected, above all, when the microphone is in direct contact with the skin of the subject. The voice of the reference person comprises rather higher-frequency sound and can be detected above all as airborne sound.

The device 30 for detecting sensory stimuli also comprises a device, not shown in FIG. 1, for the analog or digital storage of data which write the movements and noises detected by the sensor or sensors. Alternatively or additionally, the device 30 for detecting sensory stimuli can comprise a device for transmitting a data stream which describes the movements and noises detected by the sensor or sensors to the device 80 for producing sensory stimuli. This device for transmitting comprises, for example, a transmitter for transmitting the data stream via WLAN, WiFi, GSM, UMTS or in some other way.

The device 30 for detecting sensory stimuli also comprises one or more sensors (not shown in FIG. 1) for detecting the orientation of the device 30 for detecting sensory stimuli in the space, in particular in the gravitational field and/or in the magnetic field of the earth. Said sensor or sensors comprise, for example, one or more gyro sensors.

The device 30 for detecting sensory stimuli and/or the device 80, 60 for generating sensory stimuli further comprises one or more sensors (not shown in FIG. 1) for detecting a position of the device 30 for detecting sensory stimuli relative to the device 80, 60 for generating sensory stimuli. These sensors detect, for example, the distance of the device 30 for detecting sensory stimuli from the device 80, 60 for detecting sensory stimuli.

In the simplest case, this sensor or these sensors has a light sensor-optionally in addition to a light source or a magnetic field sensor or a sensor for detecting the field strength of a static or variable magnetic or electromagnetic field generated by the respective other device. In this case, it is optionally possible to dispense with an identification of the respective device and to interpret a mere approach of a different gene level as the approach of the other device.

The device 30 for detecting sensory stimuli also has one or more re-control lights 19 as signaling devices for the optical signaling of a status of the device 30 for detecting sensory stimuli or the common system 10. In particular, control lights 19 show, by means of color, number, brightness and/or time dependence of their illumination (for example continuous or flashing), in particular whether the device 30 is on or off, whether it is ready for operation whether data is stored or transmitted to the forward direction 80, 60 for generating sensory stimuli.

The device 80, 60 for generating sensory stimuli comprises two parts, a controller 80 and a body 60 made of an elastic material. The body 60 of an elastic material is provided for arrangement within an incubator as a mattress for a child. The body 60 has one or more bellows (not shown in FIG. 1) embedded in the elastic material. The controller 80 is a device for generating sensory stimuli by means of the body 60 and the bellows or bellows embedded in its elastic material and is provided and designed for arrangement outside the incubator.

The controller 80 outside the incubator is connected by means of a single fluid line 21 to the bellows or bellows in the body 60 within the incubator.

As described in more detail below with reference to FIG. 2, both a fluid flow and sound are transmitted from the controller 80 to the body 60 via the one and only fluid line 21. The fluid flow fills and leaves the bellows or bellows embedded in the elastic material of the body 60 and thus causes a low-frequency lifting and lowering of the lying surface in accordance with the breathing movement of the reference person. The sound transmitted in the fluid filling the fluid line 21 is converted into structure-borne sound in the elastic material in the bellows embedded in the elastic material of the body 60. A child lying on the lying surface of the body 60 can hear both the vertical movements of the lying surface and over its skin the structure-borne sound in the body material and, via its ears, the airborne sound produced on the surface of the body from the structure-borne sound.

The controller 80 has on its upper side a holder 20 for holding a mobile telephone or another portable computer. In the illustrated example, this holder is designed as a trough or groove into which a mobile telephone can be placed in an approximately vertical orientation.

Furthermore, the controller 80 has a signal input 22 as a receiving device for receiving an analog or digital signal from the mobile telephone or from another signal source. Via the signal input 22, the control device can receive, for example, a signal which presents the voice of the reference person or music. The data contained in the signal are converted into sound by the controller 80 by means of the body 60 and the bellows or bellows embedded in the elastic material thereof.

Alternatively or additionally, the controller 80 can also receive data for moving the reference person, in particular the upper body or the chest of the reference person, via the signal input 22. These data are set by the controller 80 by means of the body 60 and the bellows embedded in its elastic material into a vertical movement of the lying surface of the body 60.

FIG. 2 shows a schematic axonometric representation of devices in the interior of a housing of the controller 80. The controller 80 comprises a comparatively large-volume control bellows 81, the volume of which can be changed by means of a drive 83. The drive 83 comprises, for example, a spindle drive, the spindle of which is driven by means of a stepping motor.

The controller 80 also comprises a first loudspeaker 11 as a first converter device for generating airborne sound and a second loudspeaker 12 as a second converter device for generating airborne sound. In the example shown, both loudspeakers are structurally identical. The first loudspeaker 11 is, in order to produce sound, which transports speech, music or other noises, and is controlled by means of a corresponding signal. The second loudspeaker 12 is provided for generating sound, which corresponds to heart sounds of the reference person, and is controlled by a corresponding signal.

The controller 80 further comprises a fluidic connection device consisting of a plurality of line sections 13,14,15, 16 and a plurality of T-pieces 17, 18 as pipe connection pieces. A line section 16 connects the fluid connection 78 of the control bellows 81 to a first T-piece 18. A line section 14 connects a second T-piece 17 to the first T-piece 18. The first T-piece 18 connects the control bellows 81 and the second T-piece 17 to the fluid line 21 via the line sections 16, 14. A line section 13 connects a sound funnel, the edge of which is connected in a fluid-tight manner to the membrane of the first loudspeaker 11, to the second T-piece 17. A line section 15 connects a sound funnel, the edge of which is connected to the membrane of the second loudspeaker 12 in a fluid-tight manner, to the second T-piece 17. The second T-piece connects the first loudspeaker 11 and the second loudspeaker 12 to the first T-piece and via the latter to the fluid line 21.

A fluid line coupling 26 is provided in the fluid line 21, which enables a separation of the fluid line at or near the outer surface of the housing of the controller 80—see FIGS. 1 and 2 Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

The claims in the instant application are different than those of the parent application or other related applications. Applicant therefore rescinds any disclaimer of claim scope made in the parent application or any predecessor application in relation to the instant application. Any such previous disclaimer and the cited references that it was made to avoid, may need to be revisited. Further, any disclaimer made in the instant application should not be read into or against the parent application.

What is claimed is:

1. A device for producing sensory stimuli for a child by means of a body made of an elastic material and a bellows which is embedded in the elastic material of the body, comprising:
a first converter device for generating a fluid flow for moving a lying surface for a child;
a second converter device for generating sound in a fluid, which corresponds at least to either heart sounds or human speech or music; and
a fluid line for simultaneously conducting the fluid flow generated by the first converter device and transmitting the sound generated by the second converter device to a bellows in a body made of an elastic material.

2. The device of claim 1, further comprising:
a fluidic connection device comprising three connections for simultaneously connecting the first converter device and the second converter device to the fluid line;
wherein a first connection of the fluidic connection device to the first converter device, a second connection of the fluidic connection device to the second converter device and a third connection of the fluid connection device are fluidically connected to the fluid line.

3. The device of claim 2, wherein the fluidic connection device comprises a pipe connection piece with three connections.

4. The device of claim 3a, wherein the fluidic connection means further comprises:
a first line section which connects the first converter device to a first connection of the pipe connection piece; and
a second line section which connects the second converter device to a second connection of the pipe connection piece arranged symmetrically to the first connection of the pipe connection piece;
wherein a third connection of the pipe connection piece is connected to the fluid line.

5. The device of claim 1, wherein:
the first converter device comprises an electromotively driven bellows; and
the second converter device comprises a loudspeaker.

6. The device of claim 5, wherein the loudspeaker comprises an electromagnetically or electrostatically or electrostrictive membrane, an edge of which is connected in a fluid-tight manner to an edge of a sound funnel.

7. The device of claim 1, further comprising:
a third converter device for generating sound in a fluid which generate sound corresponding to at least one of human speech and music,
wherein the second converter device is configured to generate sound corresponding to heart sounds; and
wherein the fluid line is provided and formed for simultaneously conducting the fluid flow generated by the first converter device and transmitting the sound generated by the second converter device and transmitting the sound generated by the third converter device.

8. The device of claim 7, further comprising:
a fluidic connection device with four connections for simultaneously connecting the first converter device and the second converter device and the third converter device to the fluid line;
wherein a first connection of the fluidic connection device to the first converter device, a second connection of the fluidic connection device to the second converter device, a third connection of the fluidic connection device to the third converter device, and a fourth connection of the fluidic connection device are fluidically connected to the fluid line.

9. The device of claim 8, wherein
the fluidic connection device comprises a first pipe connection piece and a second pipe connection piece; and
the first pipe connection piece connects the first converter device and the second pipe connection piece to the fluid line, and the second pipe connection piece connects the second converter device and the third converter device to the first pipe connection piece.

10. The device of claim 1, further comprising:
a body made of an elastic material;
a lying surface for a child on the body; and
a bellows embedded in the elastic material of the body,
wherein the bellows is configured to generate a vertical movement of the lying surface and to transfer sound from a fluid into the elastic material of the body; and
wherein the bellows configured to be connected to the converter devices through the fluid line.
